(19) United States
(12) Patent Application Publication
(10) Pub. No.: US 2004/0039410 A1
(43) Pub. Date: Feb. 26, 2004

(54) High-strength balloon with tailored softness

(76) Inventor: Brooke Ren, Maple Grove, MN (US)

Correspondence Address: FISH & NEAVE, 1251 AVENUE OF THE AMERICAS, 50TH FLOOR, NEW YORK, NY, 10020-1105, US

(21) Appl. No.: 10/227,554

(22) Filed: Aug. 22, 2002

(51) Int. Cl. A61M 29/00

(52) U.S. Cl. 606/192

(57) ABSTRACT

A balloon and novel method of making and using the balloon is provided. In the method, a two-step forming process is provided, which overcomes the traditional brittleness exhibited by polymers when used below Tg. Selection criteria for polymeric materials that may yield medical balloons with desirable balloon strength and flexibility when processed according to the invention is also provided.

### BACKGROUND OF THE INVENTION

This invention relates to balloons, balloon catheters, and methods of making balloons or balloon catheters that are useful in medical dilatation procedures. In general, medical dilatation procedures open obstructed blood vessels or expand medical devices, and are often done in either small body passageways or with very small medical devices. To ensure adequate dilatation and robustness in such procedures, the balloon or balloon catheter should have high strength and an extremely thin wall for flexibility and a low profile.

Essentially, a balloon catheter is a thin, flexible length of tubing having a small inflatable balloon at a desired location along its length, such as at or near its tip. Historically, a variety of materials have been used to make balloon catheters. Finding balloon materials, however, that offer high strength, flexibility, toughness, and predictable size under inflation pressure has been a challenge.

Polyethylene terephthalate (PET), for example, is a well known non-compliant material that can be used to make strong and rigid balloon catheters. The PET balloon's strength arises from the polymer's structure and high molecular orientation resulting from processing. Although PET balloons possess especially high tensile strength and tightly controllable inflation characteristics, they also have several undesirable properties. For instance, the material's stiffness makes it difficult to fold the balloon. Furthermore, PET balloons have a tendency to form pin holes or other signs of weakening, which make them easy to rupture.

Other materials, such as polyvinyl chlorides (PVC) and cross-linked polyethylene (PE) have been used to make balloon catheters. PE is known to be a semicrystalline polymer that crystallizes very quickly. The secondary intermolecular bonds between the PE chains are rather weak however, and are predominantly van der Waals forces. Balloons made from materials, such as PVC or PE, are often referred to as “compliant” because they grow in volume or stretch with increasing pressure until they break. These types of compliant polymeric materials have a relatively low yield point. The yield point of a material is defined as the stress point at which the individual molecular chains move in relation to one another such that there is a permanent deformation of the polymer structure. The distortion of the molecular chains makes it difficult to later predict balloon size as a function of pressure when the balloon is re-inflated. Consequently, these types of compliant materials are not suitable for high dilatation force balloon applications.

Polymers having strong intermolecular hydrogen bonds, such as polyamide (nylon), have received positive recognition for use in balloon applications. However, as discussed in “Nylon Plastics Handbook,” Chapter 10.5, Moisture Absorption, Dimensional Stability and Density, pp. 323-333 (Hanser/Gardner Publications 1995), incorporated herein by reference, a careful selection of the polyamide is required to avoid hydrolysis of the hydrogen bonds.

Little attention has been given to methods of forming balloons made of polymer having fast nucleation and high crystallinity, such as polybutylene terephthalate (PBT) because of the difficulty of forming balloons made from such materials. While polymeric materials that have fast nucleation and high crystallinity exhibit great mechanical strength, they also have been traditionally difficult to mold during the thermoforming process. It can be particularly difficult to mold, for example, an extruded tubing to make a balloon, when the tubing is thick. In PCT application WO 99/44649 (“the '649 application”), Wang et al. teach that a balloon made primarily of PBT may be formed by first, axially stretching the extruded tubing that eventually becomes the balloon while inflating the tubing at a pressure low enough to avoid radially expanding the tubing beyond its original non-distended diameter, and second, blowing the stretched tubing at a higher temperature. According to the '649 application, adding small amounts of boric acid to PBT improves extrusion clarity and processability by reducing crystallinity after extrusion. Zhang et al. in PCT application WO 02/26308 (“the '308 application”) teach that defects in polymeric materials, such as PBT, PET, and polyamides, may be reduced by post-extrusion modification. The modification includes, first, axially stretching and radially expanding the extruded tubing that eventually becomes the balloon, and second, blowing the stretched tubing at an elevated temperature. The '308 application further teaches that by using the post-extrusion modification a balloon may be tailored to obtain different balloon diameters when starting from a given wall thickness of extruded tubing. Neither reference, however, teaches or suggests how to attain high balloon hoop tensile strength while achieving good flexibility and toughness.

In view of the foregoing, it would be desirable to provide polymeric materials and methods to make balloons with the combined properties of high strength, good flexibility, and toughness. It is desirable to have a balloon that is not only strong but also flexible.

In the deployment of medical devices such as anastomosis connectors or stents, dilatation force and deployment results are the most important consideration. It would be especially desirable to provide polymeric materials and methods to make balloons with high balloon tensile strength that can be readily molded for use in such medical devices.

### SUMMARY OF THE INVENTION

The present invention provides a group of semi-crystalline, thermoplastic polymeric materials that when processed correctly yields desirable attributes such as high hoop strength, flexibility, and puncture resistance.

It is an object of this invention to provide a method for producing a balloon, which exhibits high dilatation force, balanced bi-axial properties, and easy re-folding after dilatation.

It is a further object of this invention to provide semi-crystalline, thermoplastic, polymeric materials that exhibit fast crystallization, dipole-dipole interaction between polar functional groups, puncture-resistance, high hoop strength and elastic stress response that is advantageous for medical dilatation procedures.

It is a further object of this invention to provide a balloon made of semi-crystalline, thermoplastic, polymeric material that exhibits fast crystallization, dipole-dipole interaction between polar functional groups, and which has a glass transition temperature above human body temperature, but yet is compliant, strong, and flexible at human body temperature.

It is still a further object of this invention to provide a method for maximizing balloon material strength by optimizing the crystal morphology through extrusion and orientation using cold and hot forming. The processing steps of the invention enable fast crystallization materials, which are generally difficult to process according to prior art methods, to be made into balloons for use in medical devices.

It is still another object of this invention to provide a novel method of utilizing balloons. This method entails operating balloons below the glass transition temperature of the balloon material, while retaining the material's strength, toughness, and flexibility characteristics. Such a balloon may be comprised of semi-crystalline polymers composed of small spherulites.

These objects, as well as others, which will become apparent from the following description, are attained by forming novel balloons using the novel process of this invention from certain polymeric materials that have a fast rate of crystallization.

### BRIEF DESCRIPTION OF THE DRAWINGS

The above and other objects and advantages of the invention will be apparent upon consideration of the following detailed description, taken in conjunction with the accompanying drawings, in which like reference characters refer to like parts throughout, and in which:

FIG. 1 plots the balloon stiffness factor as a function of the temperature at which it is tested.

FIG. 2 illustrates the effect of temperature on the balloon hoop tensile strength.

FIG. 3 illustrates the effect of temperature on the rate of polymer crystallization.

### DETAILED DESCRIPTION OF THE INVENTION

The following terms and definitions are used herein:

The term “semi-crystalline polymer” refers to polymers that exhibit some degree of crystalline order (e.g., a regularly repeating arrangement of atoms in three dimensions). Semi-crystalline polymers would include, for instance, acetal, nylon, PE, polypropylene, and polyester. In contrast, amorphous polymers have polymer chains that are randomly entwined, creating a homogeneous and isotropic material in the bulk. Amorphous polymers would include, for example, polycarbonate, polystyrene, acrylonitrile-butadiene-styrene (ABS), styrene acrylonitrile (SAN), and PVC. In most polymers that form crystals, the crystallinity is almost never perfect in the bulk. The crystalline segments are usually interspersed with linking random-conformation chains. Thus, even a polymer, such as polyethylene, which shows a high degree of crystallinity, contains non-crystalline regions, and may be referred to as a semi-crystalline polymer.

“Thermoplastics” refers to materials that become soft and moldable when heated and change back to solid when allowed to cool. Examples of thermoplastics include acetal, acrylic, cellulose acetate, nylon, polyethylene, polystyrene, vinyl, and polyester. In contrast, “thermoset” plastics, such as epoxies, phenolics, and unsaturated polyesters, develop cross links during processing. The cross linking prevents relative movements between the chains and makes the material a hard solid. Thus, heating a thermoset material degrades the material so that it cannot be re-processed satisfactorily.

“Spherulites” refers to polycrystalline structures that originate from a single crystal nucleus or defect, from which lamellar fibrils may grow radially.

In polymers that may have more than one glass transition temperature, the term “glass transition temperature” shall refer to the lowest glass transition temperature displayed by the material.

“Cold forming” refers to a process of deforming a polymeric tubing by axially stretching and optionally radially expanding with internal pressure the tubing while below its glass transition temperature.

“Thermoforming” refers to a process of producing plastic parts under pressure and elevated temperature (e.g., above the polymer's glass transition temperature).

A balloon is “compliant” if it is able grow in volume and stretch at least 5% beyond its non-distended balloon diameter at 200 psi. The “non-distended balloon diameter” corresponds to the nominal diameter of the balloon.

The stiffness of a balloon may be quantified by a “stiffness factor.” As described by G. Grover, M. Sultan, and S. Spivak in “A Screening Technique for Fabric Handle,” JTI, Vol. 84(3),T486 (1993), the stiffness factor of a balloon may be measured by attaching the balloon to a Chatillon force gauge and pulling it through a hole. The stiffness factor reported herein is measured at ambient temperature (i.e., 22° C.) using a 2 mm hole (approximating the diameter of an anastomosis connector) with a balloon that has been deflated and which has a non-distended diameter that is about 3 mm. The stiffness factor is calculated by dividing the pull-through force (measured in pounds) by twice the thickness of the balloon wall (measured in inches). The higher the calculated stiffness factor number, the stiffer the balloon. This test method is commonly referred to as a “ring test,” and has been widely used, as shown in “Measuring Film Stiffness,” Modern Packaging 2, p. 121 (1963) and “Quantitative Measurement of the ‘Feel’ of Fabric,” NASA Tech. Brief LAR-12147 (1977), to characterize thin-films and fabrics.

It should be understood that the crystallization time is a function of both nucleation rate and crystal growth rate. Polymeric materials having a “fast crystallization time” refers to materials that have a fast rate of nucleation and a slow crystal growth rate relative to the nucleation rate. The “crystallization time” is the minimum crystallization half-time at the temperature that the material crystallizes the quickest. As described in U.S. Pat. No. 5,039,727, incorporated herein by reference, the crystallization time is measured by placing a small portion of polymer on a slide glass, covering it with a cover glass, and heating the polymer until it melts. Once the polymer melts, it is cooled to the temperature that the polymer crystallizes the quickest and observed under a polarizing microscope to observe changes in the amount of light as a result of crystallization.

“Viscoelasticity” refers to the changes in a polymer's mechanical properties as a function of temperature and strain rate. Polymers described as viscoelastic can display the properties of either an elastic solid and/or viscous fluid, depending on the temperature or time scale at which the polymer is observed. More specifically, at temperatures below Tg, the polymer's behavior is predominantly elastic. At temperatures above T_{g}, however, the polymer's behavior is predominantly viscous. In addition to the temperature, the rate a polymer is deformed (e.g., strain rate) can significantly affect the mechanical properties exhibited by a polymer. For instance, reducing the rate that a polymer is deformed can cause the polymer to demonstrate a viscous behavior similar to that exhibited by the same polymer at higher temperatures.

In the transition region between the elastic state and viscous state, a polymer is in a “leathery state.” The leathery state of a polymer refers to a state near the glass transition temperature in which the mechanical behavior of the polymer becomes sluggish. In this leathery state, the behavior from both the elastic and viscous states significantly contributes to the mechanical response of the material. Although a polymer in a leathery state can be extensively deformed, it generally will slowly return to its original state when the stress is removed. In the leathery state, viscous sliding of the polymer chains occurs with difficulty, and the polymer exhibits a high elastic hysteresis.

“Secondary intermolecular bonds” refers to hydrogen bonds, dipole-dipole interactions, van der Waals interactions, and ionic interactions.

The melt index (“M.I.”) measures the rate of extrusion of thermoplastics through an orifice. The M.I., as reported herein, were measured using the ASTM D 1238 testing procedure.

“Modulus” provides a measure of a material's resistance to deformation as a function of stress. The modulus of a material is calculated by measuring the change in stress as a function of strain.

The “hoop ratio” is the ratio of the predetermined balloon diameter to the original diameter of the extruded tubing.

The hoop tensile strength of the balloons reported herein were measured at ambient temperature using the well known membrane equation:

HTS=(burst pressure (psi))×(non-distended balloon diameter)/2×(wall thickness)

where HTS is the balloon hoop tensile strength.

A description of preferred embodiments of the invention follows.

In some medical procedures, catheter balloons are needed to deliver a radial force to expand either a medical device or a blood vessel. In anastomosis connector deployment, for instance, a high internal pressure is applied to a balloon to expand a connector. To perform the procedure, the balloon should have sufficient strength to avoid bursting at high internal pressure and a predictable balloon hoop tensile strength. In addition, it is desirable that the balloon be flexible, puncture resistant, and have thin walls. Semi-crystalline polymers processed according to the present invention have been found to exhibit the desired properties that make them advantageous for high internal pressure applications.

In addition, balloons of the invention may be suited for medical applications not requiring repeated inflation of a balloon, where the balloon's ability to reach the same diameter at the same pressure during repeated inflation-deflation cycles is not critical. Polymeric materials that exhibit the ability to follow the same stress-strain curve during repeated application and relief of stress are described as having a high degree of elastic stress response. The elastic stress response may be determined according to the method described in U.S. Pat. No. 6,283,939. The larger the calculated elastic stress response, the less repeatable the diameter attained by a balloon inflated to a particular pressure. In one embodiment, the balloons of the invention exhibit an elastic stress response that is greater than 5.

As further discussed below, the morphology and crystal size of semi-crystalline polymers can heavily influence a number of the polymer's properties, including material flexibility (i.e., stiffness), strength, toughness, and glass transition temperature (T_{g}). Semi-crystalline polymers of the invention are preferably processed to form small spherulite crystal morphology. As the name implies, spherulites grow radially from a nucleation site, such as a single crystal or defect. They may be obtained from either a concentrated solution or from melt. Polymers that are melt crystallized commonly develop as spherulites. Thus, crystallizing the polymer from melt is the preferred method of crystallization in the invention. Polymers composed of small spherulites are preferred because they are usually more flexible than comparable polymers made of large spherulites, which are more difficult to fold (i.e., stiffer) and less puncture resistant. Preferably, the spherulites are smaller than about 20 microns when measured with a polarized light microscope or scattering X-ray microscope.

The morphology and extent of crystallinity obtained depends, in part, on the crystal nucleation rate and crystal growth rate. Slow nucleation and fast crystal growth tend to result in a small number of large spherulites, whereas fast nucleation and slow crystal growth tend to result more preferably in a larger number and density of small spherulites. Because spherulites tend to grow until they impinge upon another spherulite or other surface interface that interferes with further crystal growth, suitable materials for use in the invention preferably nucleate spherulites at a high enough concentration such that each spherulites does not have the ability to grow too large. Preferably, the spherulites in the invention are less than about 20 microns in the longest dimension.

Although a nucleating agent may be added to facilitate nucleation of spherulites, certain agents may prompt, upon exposure to human tissue, an undesirable reaction in the human body. Accordingly, the selected polymer preferably nucleates quickly enough so that the addition of a nucleating agent is not necessary. As demonstrated in Table 1, balloons made from materials that crystallized faster tend to exhibit greater flexibility when formed according to the invented process. In a preferred embodiment of the invention, the balloon generally has a stiffness factor of less than about 100 lbs/inch. In an alternative preferred embodiment, the balloon has a stiffness factor of less than about 75 lbs/inch.

TABLE 1
Material Nucleation Rate Effect On Balloon Stiffness Factor

| | | | M.I. | | | | Stiffness | Hoop Tensile | Crystal- |
|---|---|---|---|---|---|---|---|---|---|
| Run | Material | Material | (cm³/10 | Tubing ID | Tubing OD | Balloon | Factor | Strength | lization |
| No. | Type | Brand | min.) | (in.) | (in.) | Tg (° C.) | (lbs/inch) | (psi) | time (s) |
| 1 | PET | EASTAPAK ® | 6.8 | 0.019 | 0.042 | 61 | 140 | 32836 | 18 |
| | | 7352 | | | | | | | |
| 2 | PBT | BASF ® | 19 | 0.020 | 0.042 | 52 | 124 | 24944 | 3 |
| | | PBT4500 | | | | | | | |
| 3 | PBT | CELANEX ® | 6.5 | 0.020 | 0.042 | 49 | 88 | 26171 | 3 |
| | | 1600 | | | | | | | |
| 4 | PBT | VALOX ® | 6 | 0.025 | 0.043 | 56 | 70 | 25372 | 3 |
| | | 315 | | | | | | | |
| 5 | PTT | CORTERRA ® | 6.8 | 0.020 | 0.042 | 57 | 65 | 27196 | 10 |
| | | 200 | | | | | | | |

The rate a polymer crystallizes is influenced by a number of factors, including the length of the polymer chain, size of the side chains connected to the polymer backbone, molecular weight, and processing conditions. Because long chain polymers have order on long length-scale regions, they usually nucleate faster than shorter chain polymers, allowing for a faster crystallization time. Bulky side chains off the backbone of the polymer, however, can slow crystallization by reducing the polymer's mobility for crystal growth. Crystal growth can also be slowed by lowering the temperature or may be even quenched by reducing the temperature to below the T_{g} to stop the molecular diffusion of polymer chains to the crystal growth surface. Additional details regarding the crystallization kinetics of fast crystallization materials, such as PBT, may be found in U.S. Pat. No. 5,039,727, incorporated herein by reference. In a preferred embodiment, the polymer has a crystallization time of less than about 20 seconds. In an alternative preferred embodiment, the polymer has a crystallization time of less than about 10 seconds. In a further alternative embodiment, the polymer has a crystallization time of less than about 5 seconds. In another alternative embodiment, the polymer has a crystallization time of less than about 3 seconds.

In the invention, selecting a semi-crystalline polymer with certain functional groups that create secondary intermolecular bonds between the polymer chains is among the ways one can improve the extent to which the polymer crystallizes. Secondary intermolecular bonds can help make the polymer chains pack tighter. In addition to improving the extent and rate of crystallization, secondary intermolecular bonds, such as hydrogen bonding and dipole-dipole interactions, among functional groups can contribute to the polymer's mechanical strength. Examples of polymers that have strong attractive chain interactions include, but are not limited to, polyester, polyamide, polyurethane, polyetheretherketone, and polyimide.

As noted above, hydrogen bonding can play a significant role in the crystallization process, and in some instances, may improve the polymer's mechanical strength. Polymeric materials to be used in the two-step forming process of the invention are preferably held together in part by secondary intermolecular bonds that are weaker than typical hydrogen bonding forces (see Table 2). Most preferably, the strongest secondary intermolecular bonds of the polymer selected for use in the invention are dipole-dipole interactions between polar functional groups in the polymer. It is believed that intermolecular attraction between polymeric chains as a result of hydrogen bonding may overcome the beneficial effect of the two-step forming process. Thus, although hydrogen bonds may be present in the selected semi-crystalline polymer, the strongest secondary intermolecular bonds in the polymer are preferably and predominantly dipole-dipole interactions between polar functional groups of the polymer.

TABLE 2
Secondary Intermolecular Bond Energies

| | Distance between | Bond energy |
|---|---|---|
| Intermolecular bond | atoms (nm) | (kcal/mole) |
| Hydrogen Bonds | ˜0.2-0.3 | 3-7 |
| Dipole Interactions | ˜0.2-0.3 | 1.5-3 |
| van der Waals Forces | ˜0.3-0.5 | 0.5-2 |
| Ionic Bonds | ˜0.2-0.3 | 10-20 |

It has been discovered that a number of polymers may be good candidates for use in the invention based on their rapid crystallization and relatively high material strength, which arises in part from the dipole-dipole interaction between polar functional groups of the polymeric chain. The following is a list of example polymers that may be used in the invention: polybutylene terephthalate (PBT), polybutylene naphthalate (PBN), polytrimethylene terephthalate (PTT), and polytrimethylene naphthalate (PTN). Copolymers comprising any combinations of PBT, PBN, PTT, and PTN may also be used in the invention. Copolymers of PTT, PBN, or PTN with polyethers are further examples of materials that may be used. In an alternative embodiment, PBN may be used as a homopolymer for a single layer balloon. Other polymers not listed above may also be used in the invention provided the predominant secondary intermolecular bonds in the polymer come from dipole-dipole interaction between polar functional groups and the polymer has an adequate nucleation rate and crystal growth rate properties to result in a high density of spherulite crystals, yielding a strong and flexible polymeric material.

In accordance with the invention, high molecular weight polymers are more advantageous than low molecular weight polymers. High molecular weight polymeric balloons of the invention tend to be more flexible and have a higher hoop tensile strength than balloons made with low molecular weight polymers. As shown in Table 3, higher molecular weight polymeric balloons (reflected by a lower melt index in the last three rows of Table 3) are less crystalline (reflected by a lower enthalpy) but more flexible (reflected by a lower stiffness factor) than the low molecular weight polymeric balloon formed from the material in the first row of the table. As the molecular weight of a polymer rises, the molecular chains of the polymer can grow to such an extent that they become entangled with other polymer chains and become unable to slip along each other. This entanglement makes the polymer chains difficult to untangle, and accordingly makes the polymer stronger and more resilient to stress and strain. Unlike crystallinity, chain entanglement advantageously does not increase the material's stiffness. The M.I. corresponding to the point where the material achieves the best flexibility and hoop tensile strength may be referred to as the “entanglement point.” The entanglement point depends on the specific composition of the polymer. PBT balloons of the invention, for example, have an entanglement point at an M.I. around 10.

Above a certain molecular weight, the crystallinity and stiffness start to increase. As shown in the last two rows of Table 3, as the molecular weight further rises beyond the entanglement point, the polymer exhibits increasing crystallinity accompanied by a loss in flexibility.

TABLE 3
Molecular Weight Effect On Crystallinity and Glass Transition Temperature

| Run | Material | | M.I. | | Enthalpy | Stiffness Factor | Hoop Tensile |
|---|---|---|---|---|---|---|---|
| No. | Type | Material Brand | (cm³/10 min.) | T_{g} (° C.) | H (J/g) | (lbs/inch) | Strength (psi) |
| 6 | PBT | ULTRADUR ® | 19.0 | 38 | 52.9 | 83 | 25269 |
| | | 4500 | | | | | |
| 7 | PBT | ULTRADUR ® | 9.0 | 36 | 45.3 | 64 | 27481 |
| | | 6500 | | | | | |
| 8 | PBT | CELANEX ® | 6.5 | 42 | 47.3 | 67 | 25292 |
| | | 1600 | | | | | |
| 9 | PBT | VALOX ® 315 | 6.0 | 42 | 49 | 69 | 25732 |

Intrinsic viscosity may be used as an indicator of molecular weight. Generally, the higher the intrinsic viscosity, the higher should be the molecular weight of the polymer. In a preferred embodiment, the balloon is made from a material that has an intrinsic viscosity of between about 0.8 and about 1.5.

In addition, chain entanglement of higher molecular weight polymers limits the polymer chains' freedom of motion, which advantageously raises the T_{g} of the polymer. When the T_{g} of the polymer is above the operating temperature at which the balloon is used, the polymer is not in a predominantly elastic state. In this state, the long-range motion of the polymer chains are “frozen,” resulting in a polymer with a high modulus.

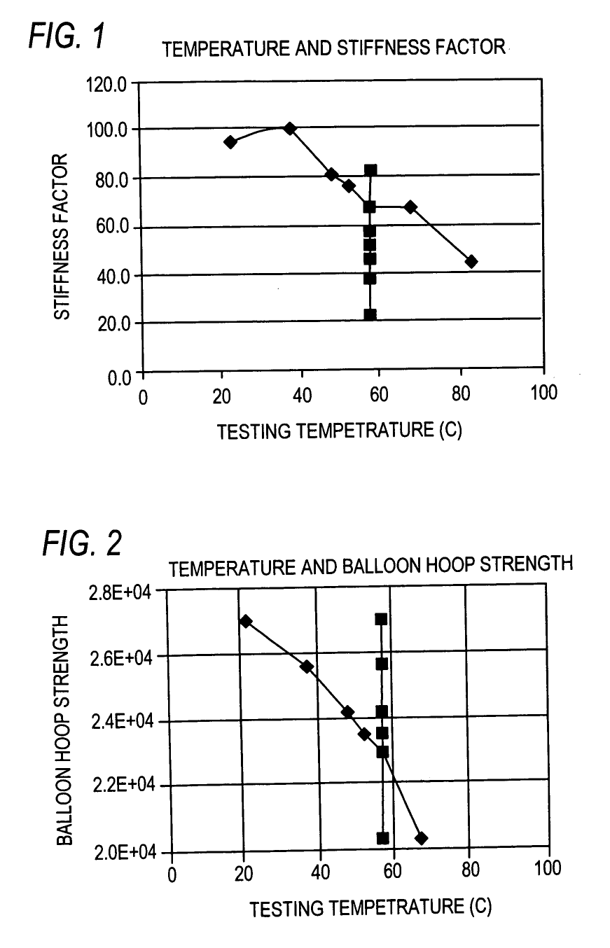

Balloons with a high modulus are preferred in the invention because they more efficiently deliver a high dilatation force under a high internal pressure. In comparison to prior-art processed balloons, such as PET balloons, which exhibit a high modulus but poor flexibility and poor puncture resistance, the balloons of the invention provide a comparable high modulus but with improved flexibility and puncture resistance as a result of the small spherulites and chain entanglement. Although decreasing the T_{g} would be another way to increase flexibility, as shown by the black data points joined by the sloping solid black line in FIGS. 1-2, the advantage gained from the improved flexibility would be outweighed by the loss of balloon hoop tensile strength.

Although the bulk properties of a polymer can differ significantly from its film properties once formed into a balloon, it has been determined that certain bulk polymers, processed according to the invention, may result in a combination of properties that are useful for making balloons to use in medical procedures. Namely, such balloons may be useful for use in medical devices requiring a high dilatation force. Bulk polymers that have a tensile strength of not less than about 5000 psi, elongation at break of not less than about 50%, and flexural modulus of not less than about 200 kpsi may produce, if processed according to the invention, balloons with high strength, good flexibility, and toughness.

In the initial balloon forming process of the invention, the selected polymer first undergoes a melt extrusion process to maximize the density of small spherulites. The formation of spherulites is favored over other crystal morphologies when the polymer molecular chains are not oriented before the melt extrusion. The polymer is preferably heated to a melt state and pumped through a die at a uniform rate to form an extruded tubing. After emerging from the die, the extruded tubing passes through an air cooling gap (e.g., tank gap). In the last step of the extrusion process, the extruded tubing is pulled by a puller into a water cooling trough (e.g., quench bath).

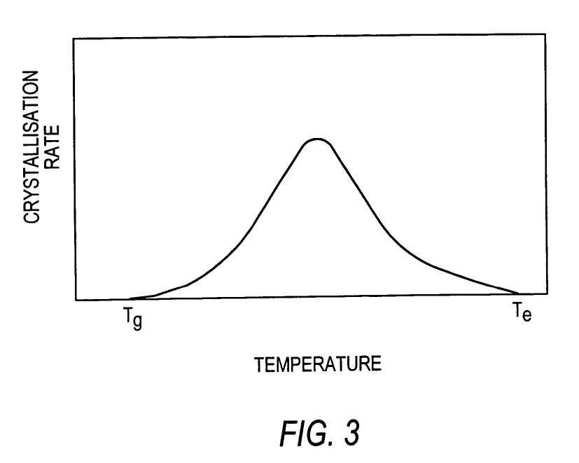

By controlling the temperature, and hence the crystal growth rate, at which the selected polymer crystallizes during the extrusion process, one can significantly affect the size and perfection of the spherulites, which can influence the polymer's melt temperature and stiffness. As shown in FIG. 3, the crystallization time of a polymer increases from T_{g} to a maximum, and then decreases as the temperature rises to the equilibrium melting point (Tₑ). The Tₑ is the temperature at which the largest, most perfect crystals can be formed by slow crystallization or annealing. The preferred temperature for initiation of nucleation during extrusion is at the peak of the curve (i.e., where crystallization is the fastest) shown in FIG. 3. The polymer preferably attains the preferred temperature in the tank gap, after emerging from the extruder but before reaching the quench bath.

By the time the extruded tubing reaches the quench bath, the majority of the nucleation should be complete. The crystal morphology may continue to grow and change, however, depending on the quenching temperature. To preserve the morphology of the small spherulites formed during the melt extrusion, the extruded tubing preferably is cooled by a quench bath that is below the T_{g} of the polymer that forms the extruded tubing. If the quenching temperature is maintained well below the T_{g} of the selected polymer, the material's crystal morphology will be essentially “frozen.” If the quenching temperature, however, is maintained just above the T_{g} of the material, then depending on the puller speed, the polymer chains in the material could have enough mobility and time to further crystallize and form a different crystal morphology, thereby making the material stiffer (see Table 4). As illustrated in Table 4, lowering the quenching temperature helps to produce a material that is more flexible. If the extruded tubing is comprised of PBT, the tubing is preferably quenched at a temperature between about 5° C. and about 40° C. In an alternative embodiment, tubing made of PTT is preferably quenched at a temperature between about 5° C. and about 50° C.

Those skilled in the art will understand that the effects of freezing the morphology after extrusion may be similarly achieved by altering the tank gap distance or die temperature.

TABLE 4
Quenching Temperature Effect On Stiffness

| | | | | | Quench | | Stiff- |
|---|---|---|---|---|---|---|---|
| Run | | | | Sample | Temp. | T_{g} | ness |
| No. | Material | Material Brand | M.I | Form | (° F.) | (° C.) | factor |
| 10 | PBT | CELANEX ® | 6.5 | extru- | 10 | 42 | 82.4 |
| | | 1600 | | sion | | | |
| | | | | tubing | | | |
| 11 | PBT | CELANEX ® | 6.5 | extru- | 38 | 42 | 87.9 |
| | | 1600 | | sion | | | |
| | | | | tubing | | | |
| 12 | PBT | CELANEX ® | 6.5 | extru- | 66 | 45 | 95.1 |
| | | 1600 | | sion | | | |
| | | | | tubing | | | |

The extruded tubing next undergoes a two-step forming process, which includes cold forming the tubing into an intermediate balloon blank and subsequently thermoforming the balloon blank into a balloon. This two-step forming process helps to further increase the density of spherulites and is particularly advantageous for polymers that have fast nucleation and high crystallinity. When processing extruded tubing according to the invention, balloons made of the appropriate material exhibit an increased balloon hoop tensile strength and, in some case, a T_{g} higher than the extruded tubing from which it came. In one embodiment, balloons of the invention have a hoop tensile strength of greater than about 20,000 psi. In a more preferred embodiment, balloons of the invention have a hoop tensile strength of greater than about 30,000 psi.

In the first step of the invented forming process, the extruded tubing is “cold formed” into an intermediate balloon blank. During the cold-forming step, the extruded tubing is axially stretched at a temperature no higher than about T_{g}+10° C. Preferably the extruded tubing is axially stretched at a temperature when the polymeric material composing the intermediate balloon blank is in its leathery state. Extruded tubing made of PBT, for instance, is preferably cold formed at a temperature between about 15° C. and about 40° C.

It is believed that the crystalline structure developed in the polymer from the extrusion undergoes a plastic deformation, causing the polymer chains to realign. The realignment imparts a strong molecular orientation to the polymeric material and helps to make the walls of the blank stronger. Parameters that may be controlled to influence the outcome of the cold-forming process include, for example, internal pressure, temperature, and deformation rate. One can alter a combination of these parameters as needed to strengthen the walls of the balloon formed according to the invention.

For instance, if the extruded tubing is inflated to radially expand its diameter during the axial stretching, the material preferably is axially stretched to at most about 300% of its original length and radially expanded by pressure to at most about 150% beyond the non-distended radial diameter of the extruded tubing.

During the cold-forming, the extruded tubing is preferably stretched at a constant linear rate no faster than about 100 inches/minute. More preferably, the material is stretched at a constant linear rate no faster than about 20 inches/minute. In an alternative embodiment, the material is stretched at a constant linear rate no greater than about 5 inches/minute. The extruded tubing is preferably axially stretched until it reaches its ductile limit. The ductile limit corresponds to the maximum plastic deformation an extruded tubing can experience before the polymer in the tubing fractures.

For a given material and intermediate balloon blank dimensions, the less the material is axially stretched, the more the material may be radially expanded during the cold-forming process. Likewise, the more the material is axially stretched, the less the material can be radially expanded. One can increase the radial expansion of the extruded tubing during cold forming by applying a high internal pressure to the extruded tubing. If a low internal pressure is used, the inner diameter of the formed intermediate balloon blank will generally be smaller than the starting diameter of the balloon blank. This is a result of the axial stretching. If the inner diameter of the intermediate balloon blank is about the same size or smaller than the starting diameter of the balloon blank, the intermediate balloon blank is preferably inflated during thermoforming by an internal pressure that is higher than that used during the cold-forming process. If a high internal pressure is used, however, during the cold-forming step (e.g., causing the tubular blank to expand beyond its original diameter), the inflation pressure used during the thermoforming is preferably lower than the pressure used during cold forming.

If the extruded tubing is not inflated during the axial stretch, then the axial deformation rate should be reduced to give the polymeric structures in the material more time to reorient. It is believed that by axially stretching the extruded tubing at a sufficiently slow rate during the cold forming, the recoil of the polymeric chains composing the tubing imparts a multi-dimensional orientational strength to the material as a result of the new polymeric crystalline structure formed during cold forming. At a given temperature, axially stretching the extruded tubing more slowly allows the polymeric material more time to relax and crystallize. The preferred stretching rate is dependent on the difference between the cold-forming temperature and the T_{g} of the polymer. Generally, the closer the temperature is to the T_{g}, the faster the preferred stretching rate should be.

For example, to cold form an extruded tubing made of PBT at room temperature (e.g., between about 20° C. and 40° C. lower than the T_{g} of PBT), the tubing is preferably stretched at a rate no greater than about 20 inches per minute. More preferably, the tubing is stretched at a rate less than about 10 inches per minute. Even more preferably, the stretching rate is no greater than about 5 inches per minute. However, at a cold-forming temperature higher than room temperature (i.e., closer to the T_{g} of PBT), the tubing comprising PBT may be stretched as fast as about 300 inches/minute.

After the cold forming first step, the intermediate balloon blank is thermoformed. In this second step, the intermediate balloon blank is heated to a temperature at least about 10° C. higher than T_{g}, while pressure is applied to expand the intermediate balloon blank. Preferably, the intermediate balloon blank is heated to a temperature no higher than about 10° C. below its melt temperature. The thermoforming step further crystallizes the material to form additional small spherulites, yielding a balloon that advantageously exhibits balanced material orientational properties, including a balanced burst failure mode.

Preferably, the intermediate balloon blank is expanded during thermoforming to no more than about 800% of the original non-distended radial diameter of the tubular blank. More specifically, the intermediate balloon blank is preferably expanded to a hoop ratio that yields a balloon with the highest hoop tensile strength.

An intermediate balloon blank made of PBT, for example, preferably would be thermoformed at a temperature between about 85° C. and about 150° C. The PBT intermediate balloon blank most preferably is expanded to attain a hoop ratio of about 5.6.

The two-step forming process advantageously preserves the small spherulites formed during the extrusion process and helps promote additional small spherulite formation in the polymer material. The small spherulites made during the extrusion and the two-step forming process helps produce a compliant balloon with balanced biaxial material properties and improved balloon hoop tensile strength. This results in a balloon that exhibits the ability to impart a high dilatation force and easy re-folding after dilatation.

In order that this invention be more fully understood, the following examples are set forth. These examples are for the purposes of illustration and are not to be construed as limiting the scope of the invention in any way.

### EXAMPLES

### Example 1

The following examples illustrate that the two-step forming process enables forming balloons that have been traditionally difficult to thermoform. As shown in Tables 5(a)-(b), the two-step forming process also imparts higher balloon hoop tensile strength than prior art forming processes.

In run number 13 of Table 5(a), a PBT balloon was made without the cold-forming step. The balloon in run number 14 was cold formed by stretching the tubing at ambient temperature in air. As shown in run number 14, cold forming helped increase the balloon's hoop strength. Using the same moderate stretch rate as run number 14, the balloon in run number 15 was cold formed at 30° C. in a water bath, resulting in a higher balloon hoop tensile strength.

PBT balloons made from ULTRADUR® 4500 were similarly made with and without cold forming. As shown in run numbers 16 and 17, cold forming improves balloon hoop tensile strength. Because of the increased material hoop tensile strength from cold forming, the intermediate balloon blank formed in run number 17 required a greater thermoforming pressure than run number 16 to inflate the balloon blank into a balloon.

Run numbers 18 and 19 compare two different cold-forming methods using VALOX® 315. As shown in run number 19, cold forming without internal pressure may yield balloons having superior balloon hoop tensile strength compared to balloons cold formed with internal pressure. The improvement to the material tensile strength is reflected in part by the necessity to use a higher thermoforming pressure to inflate the balloon blank in run number 19 compared to run number 18.

Run numbers 20-23 in Table 5(b) compare the effects of cold forming on PTT balloons. As shown in Table 5(b), PTT balloons that underwent cold forming produced higher balloon hoop tensile strengths than a balloon made without cold forming.

The glass transition temperature of the balloons of the invention is preferably above human body temperature (i.e., above about 37° C.). More preferably, the glass transition temperature is above about 40° C. In one embodiment of the invention, the balloon has a glass transition temperature of between about 45° C. and about 60° C. Even when the glass transition temperature is above 40° C., the balloons remain compliant and suitable for use with a medical device in the human body.

TABLE 5(a)
Performance Comparison of PBT Balloons With and Without the Two-Step Forming Process

| Run No. | 13 | 14 | 15 | 16 | 17 | 18 | 19 |
|---|---|---|---|---|---|---|---|
| Material Brand | CELANEX ® | CELANEX ® | CELANEX ® | ULTRADUR ® | ULTRADUR ® | VALOX ® | VALOX ® |
| | 1600 | 1600 | 1600 | 4500 | 4500 | 315 | 315 |
| Tubing ID (in) | 0.036 | 0.036 | 0.036 | 0.036 | 0.036 | 0.025 | 0.025 |
| Tubing OD (in) | 0.045 | 0.045 | 0.045 | 0.045 | 0.045 | 0.045 | 0.045 |
| Cold Forming | | | | | | | |
| Temperature (° C.) | N/A | 22 | 30 | N/A | 22 | 22 | 22 |
| Pressure (psi) | N/A | 0 | 0 | N/A | 0 | 350 | 0 |
| Strain rate | N/A | 5 | 5 | N/A | 5 | 10 | 5 |
| (inches/min) | | | | | | | |
| Radial Expansion | N/A | −35% | −42% | N/A | −35% | 32% | −18% |
| Axial Elongation | N/A | 250% | 280% | N/A | 250% | 210% | 250% |
| Thermal Forming | | | | | | | |
| Temperature (° C.) | 95 | 95 | 95 | 95 | 95 | 95 | 95 |
| Pressure (psi) | 250 | 250 | 250 | 250 | 350 | 350 | 450 |
| Balloon | 0.00136 | 0.00014 | 0.00124 | 0.001216 | 0.00166 | 0.00166 | 0.00175 |
| diameter (in) | | | | | | | |
| Balloon hoop | 19727 | 21357 | 22282 | 27091 | 27530 | 25452 | 29771 |
| tensile | | | | | | | |
| Strength (psi) | | | | | | | |

TABLE 5(b)
Performance Comparison of PTT Balloons With and
Without the Two-Step Forming Process

| Run No. | 20 | 21 | 22 | 23 |
|---|---|---|---|---|
| Material Brand | RTP ® 4700 | RTP ® 4700 | RTP ® 4700 | RTP ® 4700 |
| Tubing ID (in) | 0.020 | 0.020 | 0.020 | 0.020 |
| Tubing OD (in) | 0.042 | 0.042 | 0.042 | 0.042 |
| Cold Forming | | | | |
| Temperature | N/A | 50 | 40 | 40 |
| (° C.) | | | | |
| Pressure (psi) | N/A | 0 | 260 | 450 |
| Strain rate | N/A | 20 | 10 | 10 |
| (inches/min) | | | | |
| Radial | N/A | −73% | −36% | −14% |
| Expansion | | | | |
| Axial | N/A | 125% | 125% | 125% |
| Elongation | | | | |
| Thermal | | | | |
| Forming | | | | |
| Temperature | 75 | 75 | 75 | 75 |
| (° C.) | | | | |
| Pressure (psi) | 250 | 300 | 250 | 270 |
| Balloon | 0.00158 | 0.00144 | 0.00145 | 0.00167 |
| diameter (in) | | | | |
| Balloon hoop | 25430 | 26854 | 28850 | 27775 |
| tensile | | | | |
| Strength (psi) | | | | |

### Example 2

Tests were performed to compare extruded tubing versus balloons made with the two-step forming process. To form the balloon, an extruded tubing made from each type of material was cold formed by radially expanding and simultaneously axially stretching the tubing. The intermediate balloon blank was subsequently thermoformed. As shown in Table 6, the balloons formed with the two-step forming process had a higher T_{g} than the extruded tubing.

TABLE 6
Illustrates Glass Transition Temperature Improvement As A Result of
the Two-Step Forming Process (With Radial Expansion During
Cold Forming)

| | Material | | Tubing | Balloon |
|---|---|---|---|---|
| Run No. | Type | Material Brand | Tg (° C.) | Tg (° C.) |
| 24 | PBT | ULTRADUR ® 4500 | 38 | 52 |
| 25 | PBT | ULTRADUR ® 6550 | 36 | 52 |
| 26 | PBT | CELANEX ® 1600 | 42 | 49 |
| 27 | PBT | VALOX ® 315 | 46 | 56 |
| 28 | PTT | RTP ®4700 | 47 | 57 |

### Example 3

Table 7 illustrates the effect on balloon hoop tensile strength of the cold-forming pressure relative to the thermoforming pressure. When the inner diameter of the cold-formed tubing is about the same or smaller than the inner diameter of the extruded tubing (run numbers 29-31), the thermoforming pressure is preferably greater than the cold-forming pressure. However, if the inner diameter of the cold-formed tubing is larger than the extruded tubing (run numbers 32-33), the cold-forming pressure is preferably higher than the thermoforming pressure.

TABLE 7
Effect Of Cold-forming Pressure And Thermoforming Pressure On Balloon Hoop Tensile Strength

| Run No. | 29 | 30 | 31 | 32 | 33 |
|---|---|---|---|---|---|
| Material type | PBT | PBT | PBT | PBT | PBT |
| Material brand | CELANEX ® 1600 | CELANEX ® 1600 | CELANEX ® 1600 | CELANEX ® 1600 | CELANEX ® 1600 |
| Tubing ID (in.) | 0.020 | 0.020 | 0.020 | 0.020 | 0.020 |
| Tubing OD (in.) | 0.040 | 0.040 | 0.040 | 0.040 | 0.040 |
| Tg of extruded tubing | 42 | 42 | 42 | 42 | 42 |
| (° C.) | | | | | |
| Cold forming | | | | | |
| Temperature (° C.) | 22 | 22 | 22 | 22 | 22 |
| Pressure (psi) | 100 | 200 | 300 | 520 | 520 |
| Stretching rate | 0.300 | 0.300 | 0.300 | 0.300 | 0.300 |
| (in/sec) | | | | | |
| Radial Expansion | −20% | −17% | 1% | 13% | 13% |
| Axial elongation | 280% | 280% | 280% | 250% | 250% |
| Thermal forming | | | | | |
| Temperature (° C.) | 95 | 95 | 95 | 95 | 95 |
| Pressure (psi) | 500 | 500 | 350 | 450 | 280 |
| Balloon Diameter | 0.00154 | 0.00153 | 0.00167 | 0.00145 | 0.00153 |
| (in.) | | | | | |
| Balloon hoop | 30776 | 29331 | 30538 | 31315 | 32789 |
| tensile strength | | | | | |
| (psi) | | | | | |
| Tg of balloons (° C.) | 49 | 49 | 49 | 49 | 49 |

### Example 4

As shown in Table 8, at a given temperature, tubing that is axially stretched at slower rates produces balloons with higher hoop strength. As shown in run numbers 34-36, the faster the axial stretching rate, the lower the hoop tensile strength of the balloon. If the tubing is axially stretched too quickly, it is believed that the polymer has insufficient time to relax, resulting in a tubing that is then highly oriented in the axial direction, thus making it difficult to produce balloons with balanced biaxial material properties that are capable of imparting a high dilatation force under a high internal pressure.

TABLE 8
Effect of axial stretching rate on balloon hoop tensile strength

| Run No. | 34 | 35 | 36 |
|---|---|---|---|
| Material | PBT | PBT | PBT |
| Trade name | CELANEX ® | CELANEX ® | CELANEX ® |
| | 1600 | 1600 | 1600 |
| Tubing ID (in.) | 0.020 | 0.020 | 0.020 |
| Tubing OD (in.) | 0.040 | 0.040 | 0.040 |
| T_{g} of extruded tubing | 42 | 42 | 42 |
| (° C.) | | | |
| Cold forming | | | |
| Temperature (° C.) | 22 | 22 | 22 |
| Pressure (psi) | 0 | 0 | 0 |
| Stretching rate (in./ | 0.5 | 2 | 5 |
| min.) | | | |
| Axial elongation | 280% | 280% | 280% |
| Thermal forming | | | |
| Temperature (° C.) | 95 | 95 | 95 |
| Pressure (psi) | 500 | 500 | 500 |
| Balloon diameter (in.) | 0.00153 | 0.00156 | 0.00150 |
| Balloon hoop tensile | 29368 | 28437 | 28414 |
| strength (psi) | | | |
| T_{g} of balloons (° C.) | 49 | 49 | 49 |

### Example 5

As shown in Table 9, faster axial stretching rates are possible by increasing the cold-forming temperature. Although axial stretching at about 20 inches per minute is too fast at room temperature, the same stretching rate applied at higher temperatures will obtain high hoop strength balloons. It is believed that the elevated temperature permits the polymer chains to reorient from a stress-induced orientation to a thermally stable orientation, resulting in a balanced biaxially oriented material. Although increasing the cold-forming temperature may permit faster axial stretching rate, the cold-forming temperature used should be at a temperature that keeps the polymer in its leathery state.

TABLE 9
Effect of cold-forming temperature on balloon hoop tensile strength

| Run No. | 37 | 38 |
|---|---|---|
| Material | PBT | PBT |
| Trade name | CELANEX ® 1600 | CELANEX ® 1600 |
| Tubing ID (in.) | 0.020 | 0.020 |
| Tubing OD (in.) | 0.040 | 0.040 |
| T_{g} of extruded tubing (° C.) | 42 | 42 |
| Cold forming | | |
| Temperature (° C.) | 22 | 30 |
| Pressure (psi) | 0 | 0 |
| Stretching rate | 19 | 19 |
| (in./min.) | | |
| Axial elongation | 280% | 280% |
| Thermal forming | | |
| Temperature (° C.) | Not able to make a | 95 |
| Pressure (psi) | balloon because of | 500 |
| Balloon diameter (in.) | the high strain rate | 0.00150 |
| Balloon hoop tensile | during cold forming | 33391 |
| strength (psi) | | |

### Example 6

The two-step forming process was found beneficial especially for fast crystallization materials. Table 10 demonstrates that when the process is applied to a material that has a slow crystallization time, such as PET, neither the T_{g} nor the balloon hoop tensile strength improves in comparison to a balloon made according to a typical prior art method (e.g., run number 39).

In run number 40, a moderate stretching rate and an internal pressure of 110 psi was applied to the extruded tubing during cold forming. In run number 41, a low stretching rate and no internal pressure was used during cold forming. For comparison, in run number 42, a high stretching rate and no internal pressure was applied during cold forming. Although a longer relaxation time was used in run number 41, the balloon showed lower balloon hoop tensile strength compared to run number 42. This result is believed to arise from stress-induced crystallization caused by the faster rate of stretching the PET.

Run number 41, however, shows the most flexibility compared to the runs that do not use internal pressure during the cold-forming process. It is believed that if the crystallization rate of the materials is too slow relative to the rate the material is stretched, new additional spherulites may not have enough opportunity to form during the stretching process of the cold-forming step, resulting in a material that is stiffer.

In all of the tested PET balloons, the T_{g} of the balloon was found to be lower than that of the extruded tubing from which it was formed. Furthermore, no conditions for the two-step forming process were found to yield a balloon with better hoop tensile strength and flexibility than a balloon prepared according to known prior art conditions (run number 39).

TABLE 10
Compares Glass Transition Temperature and Balloon Hoop Tensile Strength of a Slow Crystallization Polymer

| Run No. | 39 | 40 | 42 | 42 |
|---|---|---|---|---|
| Material | PBT | PBT | PBT | PBT |
| Trade name | EASTAPAK ® 7352 | EASTAPAK ® 7352 | EASTAPAK ® 7352 | EASTAPAK ® 7352 |
| Tubing ID (in.) | 0.019 | 0.019 | 0.019 | 0.019 |
| Tubing OD (in.) | 0.042 | 0.042 | 0.042 | 0.042 |
| T_{g} of extruded tubing | 72 | 72 | 72 | 72 |
| (° C.) | | | | |
| Cold forming | | | | |
| Temperature (° C.) | 92 | 92 | 92 | 92 |
| Pressure (psi) | 0 | 110 | 0 | 0 |
| Stretching rate | 150.0 | 150.0 | 15.0 | 300.0 |
| (in./min.) | | | | |
| Axial elongation | 120% | 107% | 120% | 150% |
| Thermal forming | | | | |
| Temperature (° C.) | 95 | 95 | 95 | 95 |
| Pressure (psi) | 450 | 500 | 450 | 450 |
| Balloon diameter | 0.00135 | 0.00158 | 0.00188 | 0.00142 |
| (in.) | | | | |
| Balloon hoop tensile | 32836 | 27969 | 28632 | 30765 |
| strength (psi) | | | | |
| T_{g} of balloons (° C.) | 61 | 61 | 61 | 61 |
| Balloon stiffness | 140 | 148 | 120 | 168 |
| factor (lbs/in.) | | | | |

### Example 7

The two-step forming process was found not to be as beneficial for materials with strong hydrogen bonding, such as polyamides and nylon. As shown in Table 11, when the two-step forming process of the invention was applied to Nylon 12, for instance, and compared against the same material that underwent only the thermoforming step, neither the T_{g} nor the balloon hoop tensile strength showed improvement. As shown in Table 12, because of the strong hydrogen bonding present in the polymer, the properties of balloons made of polyamide did not vary significantly with varying process conditions. Furthermore, in all cases with the nylon balloons, the balloons formed according to two-step process exhibited a lower T_{g} than that of the extruded tubing from which it came.

TABLE 11
Comparison of Balloon Hoop Tensile Strength and T_{g} of a Hydrogen-Bonded
Polymeric Material With and Without Two-Step Forming Process

| | | | | | | | Balloon hoop | |
|---|---|---|---|---|---|---|---|---|
| Run | | | Tubing | Tubing | Cold Forming | Thermoforming | tensile | T_{g} |
| No. | Material | Trade Name | ID (in.) | OD (in.) | Pressure (psi) | pressure (psi) | strength (psi) | (° C.) |
| 43 | Nylon 12 | GRILAMID ® | 0.023 | 0.036 | N/A | 450 | 34393 | 41 |
| | | L25 | | | | | | |
| 44 | Nylon 12 | GRILAMID ® | 0.023 | 0.023 | 450 | 450 | 33950 | 42 |
| | | L25 | | | | | | |

TABLE 12
Comparison of Balloon Hoop Tensile Strength and T_{g} of a Hydrogen-Bonded
Polymeric Material Made Under Varying Process Conditions

| Run No. | 45 | 46 | 47 | 48 |
|---|---|---|---|---|
| Material | Nylon | Nylon | Nylon | Nylon |
| Trade name | GRILAMID ® L25 | GRILAMID ® L25 | GRILAMID ® L25 | GRILAMID ® L25 |
| Tubing ID (in.) | 0.024 | 0.024 | 0.024 | 0.024 |
| Tubing OD (in.) | 0.036 | 0.036 | 0.036 | 0.036 |
| T_{g} of extruded tubing | 52 | 52 | 52 | 52 |
| Cold forming | | | | |
| Temperature (° C.) | 22 | 22 | 22 | 22 |
| Pressure (psi) | 0 | 450 | 0 | 0 |
| Stretching rate | 5.0 | 5.0 | 0.5 | 10.0 |
| (in./min.) | | | | |
| Axial elongation | 120% | 107% | 120% | 150% |
| Thermal forming | | | | |
| Temperature (° C.) | 95 | 95 | | |
| Pressure (psi) | 450 | 500 | 450 | 450 |
| Balloon diameter | 0.00135 | 0.00159 | 0.00160 | 0.00158 |
| (in.) | | | | |
| Balloon hoop | 34393 | 33950 | 34814 | 35455 |
| tensile strength | | | | |
| (psi) | | | | |
| T_{g} of balloons (° C.) | 41 | 42 | 42 | 42 |

### Example 8

CELANEX® 1600, a high molecular weight grade polybutylene terephthalate with an intrinsic viscosity of 1.2, was melt extruded at a temperature between about 246° C. and about 260° C. and quenched at a temperature between about 4° C. and about 38° C. The material was extruded to form an extruded tubing with an inner diameter of about 0.02 inches and an outer diameter of about 0.04 inches. Following extrusion, the extruded tubing was cold formed at about 22° C. while inflated to a pressure of about 520 psi. During the cold forming, the tubing was axially stretched 280% at an axial stretch rate of about 5 inches/minute. Next, the intermediate balloon blank was thermoformed at a temperature of about 95° C., while inflated to a pressure of about 280 psi. The blank was subjected to a hoop ratio of about 5.6, yielding a balloon with a non-distended working diameter of about 3.0 mm. The processed balloon's hoop strength was about 32,789 psi with a Tg of about 49° C. The stiffness factor of the balloon made according to these steps is about 64 lbs/inch.

### Example 9

CELANEX® 1600, a high molecular weight grade polybutylene terephthalate with an intrinsic viscosity of 1.2,,was melt extruded at a temperature between about 246° C. and about 260° C. and quenched at a temperature between about 4° C. and about 38° C. The material was extruded to form an extruded tubing with an inner diameter of about 0.02 inches and an outer diameter of about 0.04 inches. Following extrusion, the extruded tubing was cold formed at about 30° C. without internal pressure. During the cold forming, the tubing was axially stretched 280% at an axial stretch rate of about 20 inches/minute. Next, the intermediate balloon blank was thermoformed by inflating it to a pressure of about 500 psi at a temperature of about 95° C. The blank was subjected to a hoop ratio of about 5.6, yielding a balloon with a non-distended working diameter of about 3.0 mm. The processed balloon's hoop strength was about 33,391 psi with a T_{g} of about 49° C. The stiffness factor of the balloon made according to these steps is about 50 lbs/inch.

### Example 10

Contrary to the teaching by Wang et al. in PCT Application WO 99/44649, which showed that PTT balloons could not be made if PTT tubing is stretched before blow molding, tubing comprising PTT processed according to the invention, including cold forming with or without internal pressure, was found to yield balloons suitable for use in high dilatation force medical applications. Extruded tubing comprising PTT is preferably cold-formed at a temperature between about 30° C. and about 50° C. The intermediate balloon blank made by cold forming is thermoformed at a temperature between about 85° C. and about 99° C. In a further embodiment, the balloon made of PTT has an intrinsic viscosity of between about 0.9 and about 1.5, a crystallization time of less than about 15 seconds, a hoop tensile strength of greater than about 20,000 psi, and a stiffness factor of less than about 100 lbs/inch. In an alternative embodiment, the balloon made of PTT has a glass transition temperature of between about 55° C. and about 70° C.

1. A balloon for use in a medical device that comprises a semi-crystalline, thermoplastic polymer that has a fast crystallization time, dipole-dipole molecular interaction between polar functional groups of said polymer, and spherulite crystals;
wherein said balloon has a hoop tensile strength of greater than about 20,000 psi; and
said balloon is formed by extrusion, cold forming, and thermoforming to increase a density of said spherulite crystals in said balloon.

2. A balloon for use in a medical device that comprises a semi-crystalline, thermoplastic polymer that has a fast crystallization time, dipole-dipole molecular interaction between polar functional groups of said polymer, and spherulite crystals;
wherein said polymer has a crystallization time of less than about 20 seconds and is formed by extrusion, cold forming, and thermoforming to increase a density of said spherulite crystals in said balloon.

3. A balloon for use in a medical device that comprises a semi-crystalline, thermoplastic polymer that has a fast crystallization time and dipole-dipole molecular interaction between polar functional groups of said polymer;
wherein said balloon has a hoop tensile strength of greater than about 20,000 psi and a stiffness factor of less than about 100 lbs/inch;
wherein said polymer has a crystallization time of less than about 20 seconds and is formed by extrusion, cold forming, and thermoforming.

4. A balloon for use in a medical device that comprises a semi-crystalline, thermoplastic polymer that has a fast crystallization time and dipole-dipole molecular interaction between polar functional groups of said polymer;
wherein said balloon has a glass transition temperature that is above about human body temperature and a stiffness factor of less than about 100 lbs/inch.

5. The balloon according to claim 4, wherein said balloon has a glass transition temperature of above about 40° C.

6. A balloon for use in a medical device that comprises a semi-crystalline, thermoplastic polymer that has a fast crystallization time and dipole-dipole molecular interaction between polar functional groups of said polymer,
wherein said balloon has a stiffness factor of less than about 100 lbs/inch.

7. A balloon for use in a medical device that comprises a semi-crystalline, thermoplastic polymer that has a fast crystallization time and dipole-dipole molecular interaction between polar functional groups of said polymer;
wherein said balloon has a hoop tensile strength of greater than about 20,000 psi, a glass transition temperature that is above human body temperature and a stiffness factor of less than about 100 lbs/inch.

8. A balloon for use in a medical device having a hoop tensile strength of greater than about 20,000 psi, a glass transition temperature of between about 45° C. and about 60° C., a stiffness factor of less than about 100 lbs/inch, an intrinsic viscosity of between about 0.8 and about 1.5, and a crystallization time of less than about 5 seconds.

9. A balloon for use in a medical device having a hoop tensile strength of greater than about 30,000 psi, a glass transition temperature of between about 45° C. and about 60° C., a stiffness factor of less than about 100 lbs/inch, an intrinsic viscosity of between about 0.8 and about 1.5, and a crystallization time of less than about 5 seconds.

10. A balloon for use in a medical device that comprises a semi-crystalline, thermoplastic polymer that has a fast crystallization time and dipole-dipole molecular interaction between polar functional groups of said polymer;
wherein said balloon has a hoop tensile strength of greater than about 20,000 psi and a stiffness factor less than about 100 lbs/inch.

11. A balloon for use in a medical device that comprises a semi-crystalline, thermoplastic polymer that has a fast crystallization time and dipole-dipole molecular interaction between polar functional groups of said polymer;
wherein said balloon has a hoop tensile strength of greater than about 20,000 psi and a stiffness factor of less than about 100 lbs/inch;
wherein said polymer has a crystallization time of less than about 20 seconds.

12. The balloon according to claims 8 or 9, wherein said balloon comprises a semi-crystalline, thermoplastic polymer that has a fast crystallization time and dipole-dipole molecular interaction between polar functional groups of said polymer.

13. The balloon according to any of claims 1-8, 10, 11, wherein said balloon has a hoop tensile strength of greater than about 30,000 psi.

14. The balloon according to any of claims 3-11, wherein said balloon has spherulite crystals and is formed by extrusion, cold forming and thermoforming to increase a density of said spherulite crystals.

15. The balloon according to any of claims 1, 2, 6, 10, 11, wherein said balloon has a glass transition temperature that is above human body temperature.

16. The balloon according to any of claims 1, 4-7, wherein said balloon comprises a polymer having a crystallization time of less than about 20 seconds.

17. The balloon according to any of claims 1-7, 10-11 wherein said balloon comprises a polymer having a crystallization time of less than about 10 seconds.

18. The balloon according to any of claims 1-9, wherein said balloon comprises a polymer having a crystallization time of less than about 3 seconds.

19. The balloon according to claims 1 or 2, wherein said balloon has a stiffness factor of less than about 100 lbs/inch.

20. The balloon according to claims 1 or 2, wherein
said cold forming comprises axially stretching, while in a leathery state of said polymer, an extruded tubing to form an intermediate balloon blank without internal pressure and at a rate to increase hoop tensile strength of said balloon; and
said thermoforming comprises forming said intermediate balloon blank into said balloon at a temperature between the glass transition temperature and melting temperature of said intermediate balloon blank.

21. The balloon according to claim 20, wherein said extruded tubing is stretched during cold forming at a rate no greater than about 100 inches/minute.

22. The balloon according to claim 20, wherein said extruded tubing is stretched during cold forming at a rate no greater than about 20 inches/minute.

23. The balloon according to claim 20, wherein said extruded tubing is stretched during cold forming at a rate no greater than about 5 inches/minute.

24. The balloon according to claim 20, wherein said extruded tubing is quenched at a temperature below the glass transition temperature of said polymer to reduce a stiffness factor of said balloon.

25. A balloon for use in a medical device that comprises a semi-crystalline, thermoplastic polymer that has a fast crystallization time and dipole-dipole molecular interaction between polar functional groups of said polymer;
wherein said balloon has a hoop tensile strength of greater than about 30,000 psi and a stiffness factor of less than about 100 lbs/inch.

26. The balloon according to claim 25, wherein said balloon is comprised of polymer selected from the group consisting of PBT, PTT, PTN, and PBN.

27. The balloon according to any of claims 1-11, wherein said balloon comprises polymer selected from the group consisting of PBT, PTT, PTN, and PBN.

28. The balloon according to any of claims 1, 3, 7, 8, 10, 11, wherein said polymer comprises PBT; and
said hoop tensile strength is greater than about 30,000 psi.

29. A method of forming a balloon from an extruded tubing comprising a polymer for use in a medical device;
wherein said polymer is a semi-crystalline polymer that has a fast crystallization time and dipole-dipole molecular interaction between polar functional groups of said polymer; and
wherein said extruded tubing is quenched at a temperature below the glass transition temperature of said polymer to reduce a stiffness factor of said polymer.

30. A method of forming a balloon for use in a medical device comprising the steps of:
cold forming an extruded tubing while in its leathery state into an intermediate balloon blank by axially stretching said extruded tubing without internal pressure at a rate to increase hoop tensile strength of said balloon;
wherein said extruded tubing comprises a semi-crystalline polymer that has a fast crystallization time and dipole-dipole molecular interaction between polar functional groups of said polymer; and
thermoforming said intermediate balloon blank into said balloon at a temperature between the glass transition temperature and melting temperature of said intermediate balloon blank.

31. A method of forming a balloon for use in a medical device comprising the steps of:
extruding a polymeric tubing;
wherein said polymeric tubing comprises a semi-crystalline polymer that has a fast crystallization time and dipole-dipole molecular interaction between polar functional groups of said polymer; and
wherein said polymeric tubing is quenched at a temperature below the glass transition temperature of said extruded tubing to reduce a stiffness factor of said polymer;
cold forming said extruded tubing while in its leathery state into an intermediate balloon blank by axially stretching said extruded tubing at a rate to increase hoop tensile strength of said balloon; and
thermoforming said intermediate balloon blank into said balloon at a temperature between the glass transition temperature and melting temperature of said intermediate balloon blank.

32. The method of forming a balloon according to claim 31, wherein
said polymer comprises PBT;
said extruded tubing is quenched at a temperature between about 5° C. and about 40° C.;
said cold forming of said extruded tubing is at a temperature between about 15° C. and about 40° C., wherein said axial stretching is at a constant linear rate between about 5 inches per minute and about 300 inches per minute; and
said thermoforming of said intermediate balloon blank is at a temperature between about 85° C. and about 150° C.

33. A method of forming a balloon for use in a medical device comprising the steps of:
extruding a polymeric tubing;
wherein said polymeric tubing comprises a semi-crystalline polymer that has a fast crystallization time and dipole-dipole molecular interaction between polar functional groups of said polymer; and
wherein said polymeric tubing is quenched at a temperature below the glass transition temperature of said polymeric tubing to reduce a stiffness factor of said polymer;
cold forming said polymeric tubing while in its leathery state into an intermediate balloon blank by axially stretching said polymeric tubing at a rate no greater than about 100 inches/minute; and
thermoforming said intermediate balloon blank into said balloon at a temperature between about the glass transition temperature and melting temperature of said intermediate balloon blank.

34. A method of forming a balloon for use in a medical device comprising the steps of:
extruding a polymeric tubing;
wherein said polymeric tubing comprises a semi-crystalline polymer that has a fast crystallization time and dipole-dipole molecular interaction between polar functional groups of said polymer; and
wherein said polymeric tubing is quenched at a temperature below the glass transition temperature of said polymeric tubing to reduce a stiffness factor of said polymer;
cold forming said extruded tubing while in its leathery state into an intermediate balloon blank by axially stretching without internal pressure said extruded tubing at a rate to increase hoop tensile strength of said balloon; and
thermoforming said intermediate balloon blank into said balloon at a temperature between the glass transition temperature and melting temperature of said intermediate balloon blank.

35. A method of forming a balloon comprising the steps of:
extruding a polymeric tubing;
wherein said polymeric tubing comprises a semi-crystalline polymer that has a fast crystallization time and dipole-dipole molecular interaction between polar functional groups of said polymer; and
wherein said polymeric tubing is quenched at a temperature below the glass transition temperature of said polymeric tubing to reduce a stiffness factor of said polymer;
cold forming said polymeric tubing while in its leathery state into an intermediate balloon blank by axially stretching without internal pressure said polymeric tubing at a rate no greater than about 100 inches/minute; and
thermoforming said intermediate balloon blank into said balloon at a temperature between about the glass transition temperature and melting temperature of said intermediate balloon blank.

36. The method of forming a balloon according to any of claims 29-35, wherein said extruded tubing is axially stretched at a rate no greater than about 20 inches/minute.

37. The method of forming a balloon according to any of claims 29-31, 33-35, wherein said extruded tubing is axially stretched at a rate no greater than about 5 inches/minute.

38. The method of forming a balloon according to claim 29, wherein said extruded tubing is formed by a method comprising the steps of:
cold forming said extruded tubing while in its leathery state into an intermediate balloon blank by axially stretching said extruded tubing without internal pressure at a rate to increase hoop tensile strength of said balloon; and
thermoforming said intermediate balloon blank into said balloon at a temperature between the glass transition temperature and melting temperature of said intermediate balloon blank.

39. The method of forming a balloon according to any of claims 31-33, wherein internal pressure is not applied during said cold forming.

40. The method of forming a balloon according to any of claims 29-31, 33-35, wherein said balloon comprises a polymer selected from the group consisting of PBT, PTT, PTN, and PBN.

41. A method of forming a balloon comprising PBT for use in a medical device, comprising the steps of:
extruding a tubing comprising a polymer comprising PBT;
wherein said tubing is quenched at a temperature below the glass transition temperature of said polymer to reduce a stiffness factor of said tubing;
cold forming said extruded tubing while in its leathery state into an intermediate balloon blank by axially stretching said extruded tubing without internal pressure at a rate to increase hoop tensile strength of said balloon; and
thermoforming said intermediate balloon blank into said balloon at a temperature between the glass transition temperature and melting temperature of said cold-formed tubing.

42. A method of forming a balloon comprising a polymer comprising PBT for use in a medical device, comprising the steps of:
cold forming an extruded tubing while in its leathery state into an intermediate balloon blank by axially stretching said extruded tubing without internal pressure at a rate to increase hoop tensile strength of said balloon; and
thermoforming said intermediate balloon blank into said balloon at a temperature between the glass transition temperature and melting temperature of said cold-formed tubing;
wherein said polymer has an intrinsic viscosity of between about 0.8 and about 1.5.

43. A method of forming a polymeric balloon for use in a medical device comprising the steps of:
quenching an extruded tubing comprising PBT at a temperature between about 5° C. and about 40° C.;
cold forming said quenched tubing while in its leathery state into an intermediate balloon blank by axially stretching said extruded tubing without internal pressure at a constant linear rate between about 5 inches per minute and about 300 inches per minute; and
thermoforming said intermediate balloon blank into said balloon at a temperature between about 85° C. and about 150° C.;
wherein said polymeric balloon has an intrinsic viscosity of between about 0.8 and about 1.5.

44. A method of forming a balloon comprising PBT, comprising the steps of:
melt extruding a polymeric tubing comprising PBT at a temperature between about 246° C. and about 260° C.;
quenching said polymeric tubing at a temperature between about 4° C. and about 38° C.;
axially stretching said polymeric tubing by about 280% at an axial stretch rate of about 20 inches/minute, at a temperature of about 30° C., without internal pressure, to form an intermediate balloon blank; and
thermoforming said intermediate balloon blank at a temperature of about 95° C. by inflating said intermediate balloon blank to a pressure of about 500 psi.

45. A balloon having a glass transition temperature of above 45° C.,
wherein said balloon has a hoop tensile strength of greater than about 30,000 psi and a stiffness factor of less than about 100 lbs/inch.

46. The balloon according to claim 45, wherein said balloon comprises a polymer selected from the group consisting of PBT, PTT, PTN, and PBN.

47. A balloon comprising PBT having a glass transition temperature above human body temperature, and wherein said balloon has a hoop tensile strength of greater than about 30,000 psi, and a stiffness factor of less than about 100 lbs/inch.

48. A balloon consisting of PTT for use in a medical device.

49. A balloon comprising PTT for use in a medical device having a glass transition temperature of between about 55° C. and about 70° C.

50. The balloon according to claim 49, wherein said balloon has spherulite crystals and is formed by extrusion, cold forming, and thermoforming to increase a density of said spherulite crystals.

51. The balloon according to claim 49, wherein said balloon has a stiffness factor less than about 100 lbs/inch.

52. A balloon for use in a medical device comprising PTT having a hoop tensile strength of greater than about 20,000 psi;
wherein said balloon has spherulite crystals and is formed by extrusion, cold forming, and thermoforming to increase a density of said spherulite crystals.

53. A balloon for use in a medical device comprising PTT having a hoop tensile strength of greater than about 20,000 psi;
wherein said balloon has a stiffness factor less than about 100 lbs/inch.

54. A balloon comprising PTT for use in a medical device; wherein said PTT has an intrinsic viscosity of between about 0.9 and about 1.5, and a crystallization time of less than about 15 seconds; and wherein said balloon has a hoop tensile strength of greater than about 20,000 psi, and a stiffness factor of less than about 100 lbs/inch.

55. The balloon according to claim 54, wherein said balloon has a glass transition temperature of between about 55° C. and about 70° C.

56. The balloon according to any of claims 48-55, wherein said balloon has a hoop tensile strength of greater than about 30,000 psi.

57. A method of forming a balloon for use in a medical device comprising the steps of:
cold forming an extruded tubing comprising PTT while in its leathery state into an intermediate balloon blank at a temperature between about 30° C. and about 50° C.; and
thermoforming said intermediate balloon blank into said balloon at a temperature between about 85° C. and about 99° C.

58. A method of forming a balloon comprising PTT, comprising the steps of:
extruding tubing comprising PTT,
wherein said tubing is quenched at a temperature between about 5° C. and about 50° C.;
cold forming said extruded tubing at a temperature between about 30° C. and about 50° C. to form an intermediate balloon blank; and
thermoforming said intermediate balloon blank at a temperature between about 85° C. and about 99° C.

59. A single layer balloon comprising a polymer consisting of PBN for use in a medical device.

60. The balloon according to claim 59, wherein said balloon has spherulite crystals and is formed by extrusion, cold forming, and thermoforming to increase a density of said spherulite crystals.

61. The balloon according to claim 59, wherein said balloon has a stiffness factor less than about 100 lbs/inch.

62. A balloon comprising PBN for use in a medical device having a hoop tensile strength of greater than about 20,000 psi; and
wherein said balloon has spherulite crystals and is formed by extrusion, cold forming, and thermoforming to increase a density of said spherulite crystals.

63. A balloon comprising PBN for use in a medical device having a hoop tensile strength of greater than about 20,000 psi and a stiffness factor less than about 100 lbs/inch.

64. The balloon according to claim 62, wherein said hoop tensile strength is greater than about 30,000 psi.

65. A balloon comprising PTN for use in a medical device.

66. A balloon consisting of PTN for use in a medical device.

67. A balloon comprising PTN for use in a medical device having a hoop tensile strength of greater than about 20,000 psi; and
wherein said balloon has spherulite crystals and is formed by extrusion, cold forming, and thermoforming to increase a density of said spherulite crystals.

68. A balloon comprising PTN for use in a medical device having a hoop tensile strength of greater than about 20,000 psi and a stiffness factor less than about 100 lbs/inch.

69. The balloon according to claim 68, wherein said hoop tensile strength is greater than about 30,000 psi.

70. The balloon according to claims 50, 52, 60, 62, or 67, wherein
said extrusion comprises quenching an extruded tubing at a temperature below the glass transition temperature of said extruded tubing;
said cold forming comprises axially stretching, while in a leathery state of said polymer, said extruded tubing to form an intermediate balloon blank, without internal pressure and at a rate to maximize hoop tensile strength of said balloon; and
said thermoforming comprises forming said intermediate balloon blank into said balloon at a temperature between the glass transition temperature and melting temperature of said intermediate balloon blank.

71. The balloon according to any of claims 48-50, 52, 59, 60, 62, 64-67, 69, wherein said balloon has a stiffness factor of less than about 100 lbs/inch.

72. The balloon according to any of claims 1-11, 25, 26, 48-55, 59-69, wherein said balloon has a stiffness factor of less than about 75 lbs/inch.

73. The method of forming a balloon according to any of claims 30-32, 34, 38, 41-43, wherein said axial stretching rate is no greater than about 100 inches/minute.

74. The method of forming a balloon according to any of claims 38, 41-43, wherein said axial stretching rate is no greater than about 20 inches/minute.

75. The method of forming a balloon according to any of claims 38, 41-42, wherein said axial stretching rate is no greater than about 5 inches/minute.

76. The balloon according to any of claims 1-3, 50, 52, 60, 62, 67, wherein said cold forming comprises axially stretching an extruded tubing at a rate no greater than about 100 inches/minute.

77. The balloon according to any of claims 1-3, 50, 52, 60, 62, 67, wherein said cold forming comprises axially stretching an extruded tubing at a rate no greater than about 20 inches/minute.

78. The balloon according to any of claims 1-3, 50, 52, 60, 62, 67, wherein said cold forming comprises axially stretching an extruded tubing at a rate no greater than about 5 inches/minute.

79. The method of forming a balloon according to claims 57 or 58, wherein said extruded tubing is stretched during cold forming at a rate no greater than about 100 inches/minute.

80. The method of forming a balloon according to claims 57 or 58, wherein said extruded tubing is stretched during cold forming at a rate no greater than about 20 inches/minute.

81. The method of forming a balloon according to claims 57 or 58, wherein said extruded tubing is stretched during cold forming at a rate no greater than about 5 inches/minute.
